(19) United States
(12) Patent Application Publication
(10) Pub. No.: US 2024/0293009 A1
(43) Pub. Date: Sep. 5, 2024

(54) TETHER GUIDE

(71) Applicant: OMNISCIENT IMAGING, INC., Tucson, AZ (US)

(72) Inventors: Romit Banerjee, Tucson, AZ (US); Veysi Malkoc, Tucson, AZ (US)

(21) Appl. No.: 18/115,973

(22) Filed: Mar. 1, 2023

(51) Int. Cl. A61B 1/00

(52) U.S. Cl. CPC A61B 1/00154; A61B 1/041

(57) ABSTRACT

A tether guide that includes a plate with a peripheral edge surface defining a slit that un-interruptingly extends from a peripheral portion of the plate to its central portion thereof, and a post affixed to a first side surface of the plate to protrude from its transversely . The post includes a channel extending throughout the post along an axis to fluidly connect the first and second ends of the post. The slit merges with and ends at a bottom of the channel such that an axis of the slit is substantially transverse to the axis of the post. The method of using the tether guide.

### CROSS-REFERENCE TO RELATED APPLICATIONS

This application incorporates by reference a disclosure of Design patent application Ser. No. 29/885,831 filed on Mar. 1, 2023. This patent application also incorporates by reference the disclosure of U.S. patent application Ser. No. 17/735,446 filed on May 3, 2022 and now published as US 2022/0334454.

### TECHNICAL FIELD

The present invention relates to structures configured to support and/or guide a tether upon longitudinal repositioning thereof and, more particularly, to a contraption supporting and/or guiding a thin tether (carrying a capsule enclosing a miniaturized optical imaging system) and configurable as a mouth guard.

### RELATED ART

In order to examine specific bodily organs such as the esophagus, stomach, or small intestine (gastrointestinal tract), related art currently utilizes endoscopes—elongated, flexible or rigid, instruments configured for imaging internal bodily organs. The structures (and, in particular, the sizes and shapes) of such instruments (about 10 mm in diameter by about 1,100 mm in length) require that examination procedure be formatted in a very specific way: a patient must be at a minimum unperturbed and, preferably, sedated during the procedure. An instrument of such a large size has to be advanced from the mouth, via the pharynx to the esophagus, stomach and beyond by pushing the device forwards while viewing the path along which the device is being moved. At least a portion of such device includes a rigid or flexible long insertion sheath (typically, of about 10 mm in diameter) carrying various wires, optical channels, other hardware components in its hollow and protecting these contents from the environment.

Furthermore, a physician needs to somewhat forcibly advance the endoscope along the digestive tract; the device cannot be simply swallowed and advanced without applying this force. The operation of a so-conventionally-structured scope is then inevitably associated with physical rotation or deviation or bending of, for example, the distal end of the sheath from or with respect to its original position. That, in turn, leads to physical impact between the probe and the physical tissue, thereby causing trauma to the bodily cavity. Even to tolerate such a procedure—let alone to have the examination go smoothly and without disruptions, to collect required data—a patient typically has to be sedated, which increases the risk of a procedure by blunting the protective physiological reflexes. (See, for example, northhillsendoscopy.com/procedure/upper-endoscopy.) Traditional endoscopes achieve viewing in a plurality of directions by moving/bending the tips of the endoscopes to direct imaging cameras, which can result in trauma to the lining of an organ with which the tip interacts. Moreover, retroflexed views through such traditional endoscopes are at least partially obscured by the very sheath of the device.

To minimize trauma caused to the patient at least to some degree, various mouth guards (also referred to interchangeably as mouth pieces or bite blocks) are used (typical representatives 100, 200 of which are shown in FIGS. 1 and 2). These are typically plastic contraptions that in use are inserted in the mouth of a user to keep it ajar (and kept in place with the user's teeth) while the endoscope is inserted (once the sedation takes effect) through the main opening 110, 210 of a given mouth guard. The main opening 110, 210 is large enough to not only accommodate the substantial diameter of the sheath/cable of the endoscope but also to allow for substantial lateral movement of the sheath/cable when handled by the physician. To this end, while a conventionally-structured mouth guard transversely support the sheath/cable of conventional endoscope, it is not structured to maintain the longitudinal orientation of the sheath/cable substantially unchanged during the passing of such sheath/cable through the main opening 110, 220: the dimensions of the main opening accommodate and permit for physical rotation and/or deviation and/or tilting and/or bending of the portion of the sheath/cable passing through it that are caused by the physician's handling of the endoscope.

The conventionally-structure mouth guard protects, on one hand, the endoscope from the patient's teeth and, on the other hand, protects the teeth from possible chipping or breaking upon the impact with the endoscope (and hence is interchangeably referred to as a bite block) without interfering with breathing of the patient. (See FIG. 3). During the procedure with the use of a conventional endoscope, in addition to optical images transmitted from a camera at the distal end of the endoscope through at least one optical channel within the sheath, the clinician may additionally obtain a biopsy through the appropriate channel concealed within the sheath. At the end of the examination, the endoscope is slowly withdrawn through the opening 110, 210.

Conventionally used in related art bite blocks for endoscopy are large and keep the upper and lower teeth substantially open to allow an endoscope to pass through in-between. (As was already mentioned above, endoscopes are typically 10-12 mm in diameter along their entire length.) Standard endoscopic bite blocks keep the teeth separated by a distance large enough for a wide, cylindrical and flexible endoscope to pass.

The use of a recently disclosed novel imaging construct (one of the applications of which can be endoscopy) that is devoid of a conventional sheath and/or cable and includes, instead, only a thin tether (see US 2022/0334454) does not require or even justify the use of a conventionally-structure mouth guard at least due to completely different, minimized geometrical sizes of both the tether and the capsule-like shaped imaging camera apparatus attached to an end of the tether. Furthermore, with such minimized dimensions the problem of protection of patient's teeth is not as critical anymore, while the challenges of protecting the thin tether (which—unlike the thick sheaths of the conventional endoscopes—can be bitten through, severed if not appropriately protected) and guiding it through the opening of the mouth without substantial deviations sideways to at least facilitate the swallowing of the capsule at the end of the tether becomes the primary task. As a skilled person will readily understand, the conventional mouth guard structures cannot accommodate at least one of these requirements.

### SUMMARY OF THE INVENTION

An embodiment of the present invention provides an article of manufacture that includes a plate of a first material having a peripheral edge surface that defines a slit in the plate (here, the slit is un-interruptingly extending from a peripheral portion of the plate to a central portion thereof) and a post of a second material affixed, at a first end of the post, to a first side surface of the plate at the central portion thereof to protrude from the plate substantially transversely thereto. The post contains a channel formed throughout the post along an axis of the post and fluidly connecting the first end of the post and a second end of the post opposing the first end. The slit merges with and ends at a bottom of the channel such that an axis of the slit is substantially transverse to the axis of the post. In at least one embodiment, such channel has a cross-section in a plane that is transverse to the axis (and the cross-section includes a first portion defined by two lines that are substantially parallel to one another and separated from one another by a first distance. In at least one embodiment, the cross-section may include a second portion defined by an incomplete circle having a diameter that is larger than the first distance—in this case, as viewed in the cross-section, the overall channel looks bottle-shaped. Alternatively or in addition, a combination of the slit and the post may be configured to fluidly connect the second end with a second surface of the plate; and/or a plane containing the axis of the post and the axis of the slit may be chosen to be substantially normal to a perimeter of the plate. (In the latter case, embodiment of the article becomes substantially symmetric with respect to a plane in which both the axis of the post and the axis of the list are.) Alternatively or in addition, the post may include a ridge extending radially from the axis of the post at the second end. Substantially in every implementation, the article of manufacture may contain at least one opening therethrough and/or contain first and second of such openings that are located substantially symmetrically with respect to the post. Alternatively or in addition—and substantially in every implementation—the plate may be bent in a plane containing the axis of the post such that the post extends from the plate radially towards a center of curvature of a corresponding bend; and/or the first material and the second material may be the same and/or the plate and the post may be co-molded. In at least one case, the article may be configured as a mouth guard or bite guard (for example, for use during an endoscopy procedure) and/or possess first and second means configured to assist in securing the article, inserted in a mouth of a user, with respect to a head of the user (optionally, the first means and the second means may be configured as hooks for securing an elastic element.

Embodiments of invention also provide a method that includes a step of passing the tether through a slit of substantially every implementation (of the article of manufacture) outlined above and a step of positioning the tether along an axis of the post and in a channel of a post of the article of manufacture such that a first end of the tether is placed outside and next to a free end of the post while a second end of the tether is separated from the first end at least by a length of the post and a plate of the article of manufacture to which another end of the post is affixed. In at least one case, the step of passing may include moving the tether along an axis of the slit towards the axis of the post and/or the embodiment may additionally include a step of repositioning the tether within the channel along the axis of the post to vary axial separation between the first end of the tether and the free end of the post while substantially not changing angular orientation of a portions of the tether contained within the channel. Alternatively or in addition—and substantially in every implementation of the method—the step of positioning may include positioning the tether along the axis of the post and in the channel of the post such that an optical camera at the first end of the tether is placed outside and net to the free end of the post while a control device (cooperated with the second end of the tether) remains separated from the optical camera at least by the length of the post and the plate. Alternatively or in addition, substantially every implementation of the method may include a step of transmitting a fluid through at least one of the openings formed in the plate of the article of manufacture from a side of the plate corresponding to the second end of the tether to a side of the plate corresponding to the first end of the tether.

### BRIEF DESCRIPTION OF THE DRAWINGS

The invention will be more fully understood by referring to the following Detailed Description of Specific Embodiments in conjunction with the Drawings, of which:

FIG. 1 is an image of an Olympus® MB-142 pre-owned OEM mouthpiece (see www.endoscopy.md/items/details/19161;

FIG. 2 is an image of an endoscopy mouth guard (available at www.indiamart.com/proddetails/endoscopy-mouth-guard-6961818848.html).

FIG. 3 is a schematic illustration of the use of a typical clinical endoscope (which, in practice, is supplemented with the use of a mouth guard).

FIG. 4 is a first perspective view of an embodiment of the invention.

FIG. 5 is a second perspective view of the embodiment of FIG. 4.

FIG. 6 is a front elevational view of the embodiment of FIG. 4, indicating optional structural features.

FIG. 7 is a rear elevational view of the embodiment of FIG. 4, indicating optional structural features.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another.

### DETAILED DESCRIPTION

In accordance with preferred embodiments of the present invention, methods and apparatus are disclosed for operational support and guidance of a tether of a novel implementation of an apparatus of a tethered optical imaging camera (which in one specific case can be used for upper endoscopy, see US 2022/0334454). Such tether is intentionally dimensioned to be thin and flexible, of about 1 m in length and about a few (for example, 3) millimeters or even less in diameter, while the apparatus also includes an encapsulated optical imaging camera construct (interchangeably referred to herein as capsule, of approximately 10 mm to 15 mm wide and 20 mm long) that is attached to the distal end of the tether. The tether at least supports the exchange of electrical signals between the capsule and the proximal end of the tether (alternatively or in addition, and the apparatus may be configured to return imaging or other data signals through wires within the tether or wirelessly) and preferably does not include an optical channel (such as a lightguide) drawn through the tether in order to not limit its flexibility (which would necessarily occur otherwise, as a skilled person readily appreciates) and/or to not increase its diameter beyond a few millimeters. (While in some other related implementations that can be conceived, images may also be returned from the capsule through optical fibers contained in the tether, such implementations are not necessarily preferred.) With a flexible tether, it is not normally possible to push the capsule into the esophagus and stomach, as it is with a standard, rigid sheath endoscope. It is important if not vital for the patient to be able to swallow. Understandably, swallowing is very difficult if and when the mouth is open and widely separated by a standard bite block (which creates an opening in the mouth at least of about 15-20 mm). Therefore, for endoscopy procedures that employ an embodiment of a tethered optical imaging camera, the conventional bite block cannot be used as a supporting structure and/or guide at least because, as the teeth are widely separated, swallowing the capsule becomes practically problematic if not impossible. Additionally, while the supporting structure and/or guide for a tether of the embodiment similar to that discussed in US 2022/0334454 must not introduce a wide separation between the opposing teeth/jaws, it would still require some mechanism to enable a directional advancement of the tether into and from the mouth while maintaining a substantially unchanged orientation of that portion of the tether that passes through to facilitate the primary function of advancing the tether through the mouth (with a secondary function of protecting the tether from being bitten: a bitten tether may result in loss of function of the device, or cause an electrical shock to the patient, or detachment and loss of the capsule into the stomach or other organ being examined).

Embodiments of the invention address this need and solve the operational deficiencies of the related art that does provide the required support and guidance for the novel tethered optical imaging system. Specifically, the disclosed structure is judiciously dimensioned to possess a small-cross-section post or trunk the dimensions of which operationally limit a degree to which the jaws of the user are opened with respect to one another and which carries a longitudinally-oriented narrow channel dimensioned to substantially prevent the lateral (that is, transversely with respect to the axis of the channel and/or post or trunk) repositioning of the tether laying in the channel. At the same time, such port with the channel does not impede the movement of the tether along the channel during the delivery of the tether optical imaging system whether forwards/distally (during the delivery of the capsuled optical imaging system into esophagus and beyond) or backwards/proximally (when being withdrawn from the patient upon the conclusion of the imaging procedure).

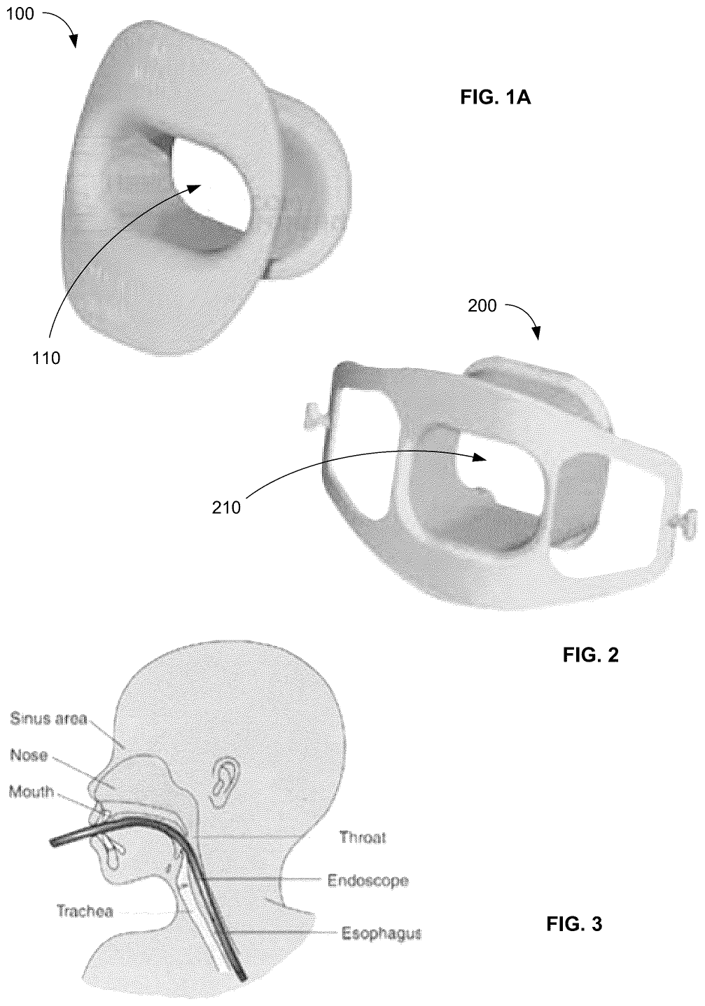

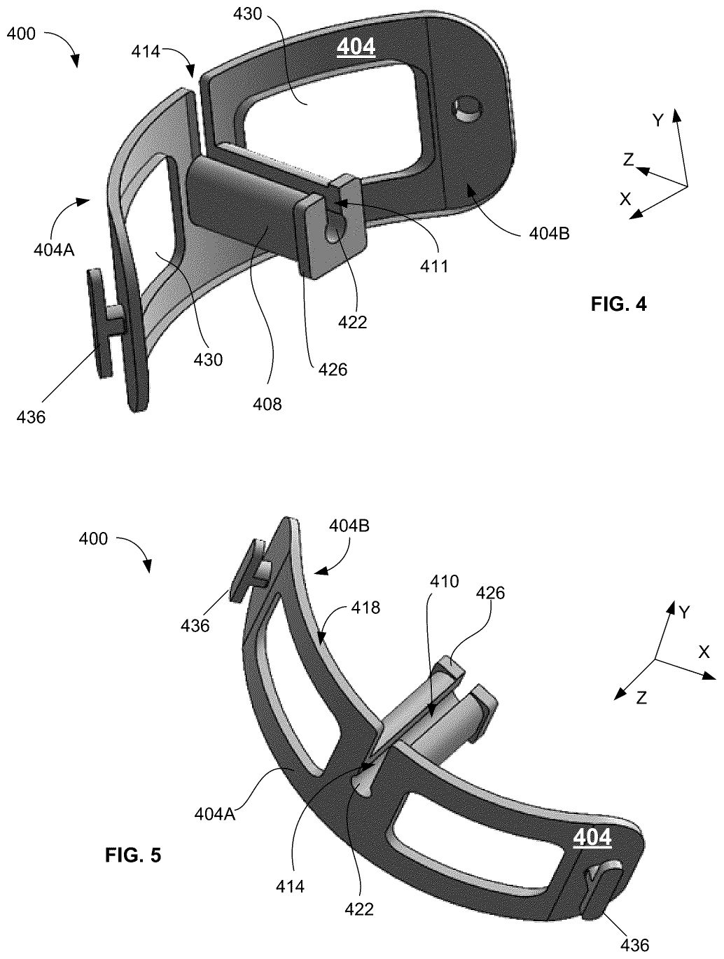

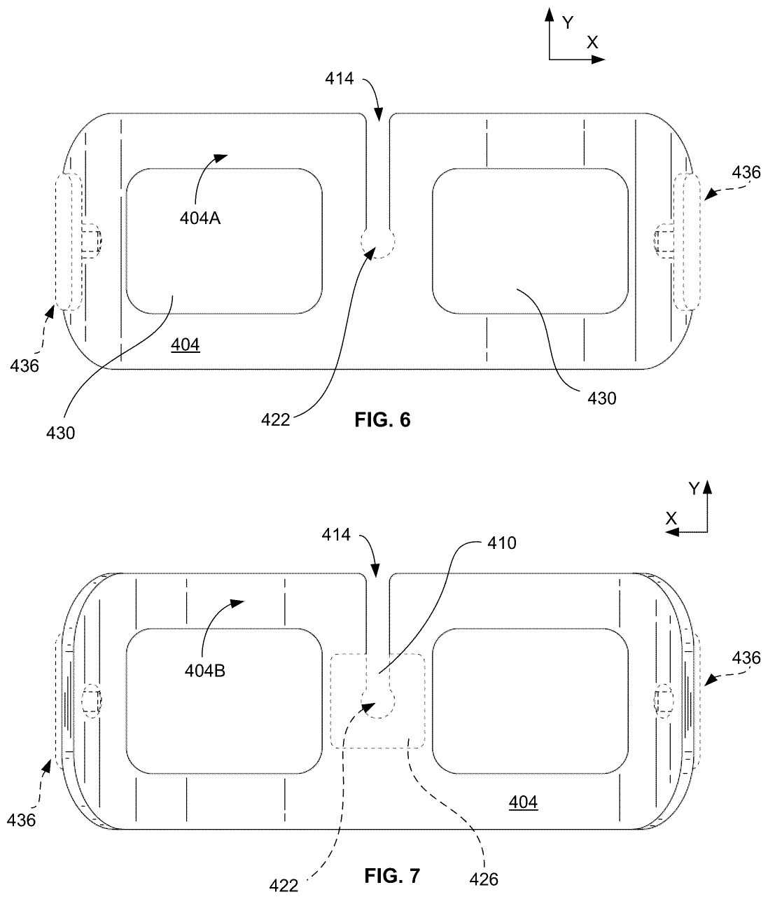

In reference to FIGS. 4 and 5, an embodiment 400 of the tether guide structure configured according to the idea of the invention includes a front plate 404 from which a preferably substantially centrally located post or trunk 408 is protruding. The length of the post is generally between 10 mm and 30 mm, preferably between 15 mm and 25 mm, more preferably between 20 mm and 25 mm, and in one specific case about 22 mm, while it's transverse dimension generally does not exceed 10 mm (in contradistinction with the dimensionality of the main openings 110, 210 of the conventional structures of FIGS. 1, 2) and is preferably between 5 mm and 10 mm, more preferably about 8 mm. In at least one specific implementation (shown here), the plate 404 may be optionally curved with a center of curvature located on the same side of the plate 404 as the post 408. The post 408 carries, longitudinally/axially, along the local z-axis, a channel 410 a transverse dimension of which (along the local x-axis) is chosen to be substantially equal to the cross-sectional dimension of a target tether (in one specific non-limiting case—the tether of an embodiment of the tethered optical imaging system disclosed in US 2022/0334454). At the same time, longitudinally or axially the channel is judiciously dimensioned such as to fluidly connect the first surface 404A of the plate 404 with the second, opposing surface 404B of the plate 404. In other words, the channel 410 axially extends throughout both the post 408 and the plate 404 (as shown in FIG. 5, for example). Notably, the plate 404 is equipped with the slit 414 directed generally transversely to the post 408 (in the specific example as shown—along the local y-axis) that “splits” the plate 404 into two wings from the very edge surface 418 of the plate down to and connecting with the channel 110 of the post 408. In one implementation, the slit 414 has substantially the same transverse dimension (across the slit 414) as the width of the channel 410.

In operation, when the plate 404 is positioned with the surface 404B abutting a predetermined support element or surface, a tether (not shown here for simplicity of illustration) is dropped through the slit 414 down to the bottom of the channel 410 and laid in the channel 410 along the z-axis such as to have the transverse and/or angular repositioning of the tether (now housed by the channel 410) in the xz-plane to be substantially limited and/or even substantially prevented regardless of whether the tether is not acted upon by the user or is being moved axially along and within the channel 410.

Generally, a foot of the post 408 is connected to the body of the plate 404, preferably at or near a center of the plate, although different positioning of the post remains within the scope of the invention. The structure of the channel 410 may be enhanced by optionally shaping a bottom portion 422 of the channel (as indicated by dashed lines in plan views of FIGS. 6 and 7) as an incomplete cylinder, the diameter of which exceeds the transverse dimension of the slit 414 and the upper portion of the channel 410 by up to 2.0 mm in general; by about 1.5 mm preferably; and by about 1.25 mm—even more preferably. This feature, while optionally allowing the tether placed at the bottom of the channel 410 to move within an extremely limited angular range, serves as a tether lock of sorts that impedes or even substantially prevents the spontaneous movement of the tether, that has been already positioned in the channel 410 and extended along and in contact with its bottom, up the channel and the slit (in the +y direction) towards the edge surface 418. The skilled person will readily appreciate that this optional feature, in stark advantageous contradistinction with related art, further enhances the capability of the embodiment of the invention to substantially uninterruptingly maintain the axial orientation of the portion of the tether located within the channel and/or—at least during the process of practical use of the embodiment 400—reducing the “element of surprise” for both the user(s) by providing a passive control of the orientation of at least a portion of the tether.

Optionally but preferably, the distal end of the post/trunk 408 (that is, the free end of the post that is separated from the plate 404 by the length of the post) can be configured to carry a ridge or rib 426 (which in one specific non limiting case is dimensioned to follow the perimeter of the outer surface of the post and be interrupted where the channel 410 is present). In the specific application where the embodiment 400 is configured to additionally perform a function of a mouth guard or a bite block—and not just as a support and guide of the tether—the presence of the ridge or rib 426 may prevent slipping of the teeth of the patient, closed about the post 408, away from the distal end of the post and onto the tether. (Typically, the ridge 426 would radially protrude over the side surface of the post 408 by about 0.1 mm to about 2.0 mm in general, preferably by about 0.2 mm to about 1.0 mm, and even more preferably from about 0.4 mm to about 0.6 mm.) If and when the embodiment 400 is configured as a mouth guard, the plate 404 is preferably additionally curved as identified above, and carries at least one throughout opening or aperture 420 dimensioned to be substantially large to accommodate a straw or a spout of a water-bottle therethrough for the user/patient to be able to sip water to facilitate the swallowing of the capsulated optical imaging system attached to the distal end of the tether while the tether is being advanced in and along the bottom of the channel 410 from the proximal end of the post 408 to the distal end of the post 408. In this specific application, the surface 404B of the curved plate 404 is abutted against the anterior lips of the patient to prevent it from entering the mouth while the post 408 is held by the wearer/patient by his teeth.

Referring again to the specific application in which the embodiment 400 also performs a function of a mouth guard, an optional but preferred anchoring feature—shown in FIGS. 4 through 6 a corresponding hook 436 (here, two T-shaped hooks) formed at each of the end zones of the front plate 400 at the surface 404a. The elastic straps having holes or loops, for example, that fit over the hooks 436 can be used to help secure the embodiment at the head of the user by, for example, being wrapped around the back of the neck or the head of the user.

Overall, embodiments of the invention provide at article of manufacture that includes a plate of a first material having a peripheral edge surface that defines a slit in the plate (where such slit un-interruptingly extends from a peripheral portion of the plate to a central portion of the plate) and a post of a second material affixed, at a first end of the post, to a first side surface of the plate at the central portion thereof to protrude from such first side substantially transversely thereto (here, the post contains a channel formed throughout the post along an axis of the post and fluidly connecting the first end of the post and a second end of the post opposing the first end and/or the first end of the post and a second side surface of the plate opposing the first side surface. The slit merges with and ends at a bottom of the channel such that an axis of the slit is substantially transverse to the axis of the post.

Understandably—and notwithstanding that the discussion of the idea of the invention was presented above in reference to and in specific context of an endoscopic procedure and/or imaging of a biological target—generally the embodiment of such article of manufacture is employed for advancement of the tether within and along the bottom of the channel 410 (while the plate 404 is secured in its position again or with a supporting element) and does not require or even involve the use of the embodiment in the endoscopic procedure. For example, an embodiment of the disclosed device may be used for non-destructive inspection of machinery through a small opening and the tether guide can be used to protect the cable from accidental damage. In such general case, the embodiment is simply used as a tether support and/or guide, as intended. Here, the method for use of the embodiment includes a step of passing a tether through a slit such article of manufacture, and positioning the tether along an axis of the post of the article of manufacture and in a channel of the post such that a first end of the tether is placed outside of the post on the side of the free end of the post while a second end of the tether outside of the post on the side of the second end of the post and is separated from the first end at least by a length of the post and a plate of the article of manufacture to which such second end of the post is affixed. In one case, the step of passing may include moving the tether along an axis of the slit towards the axis of the post and/or the method may additionally include repositioning the tether within the channel along the axis of the post to vary an axial separation between the first end of the tether and the free end of the post while necessarily substantially maintaining unchanged the spatial axial orientation of the portion of the tether housed within the channel. When the tether carries, at its distal end, an optical camera, the step of positioning may include positioning the tether along the axis of the post and in the channel of the post such that such optical camera s placed outside and next to the free end of the post while a control device, cooperated with the second end of the tether is separated from the optical camera at least by the length of the post and the plate.

In the case of one specific application of the discussed embodiment(s) of the article of manufacture, however—that is, in the case of use of endoscopy performed with a tethered apparatus (such as an encapsulated optical imaging system)—the embodiment is used to advance the tether as described that is positioned through the slit in the plate onto the bottom of the narrow channel of the post of the embodiment while protecting it from being bitten, as the tether longitudinally moves forwards or proximally along the channel. In this specific case, the embodiment is configured as a non-obviously structured mouth guard (bite block, bite guard) in that its geometry does not require—and, in fact, prevents the teeth of the user from being widely separated, which facilitates the process of swallowing, thereby providing a stark advantageous contradistinction from the mouth urads of related art. A large, curved front-plate of the embodiment fits in front of the lip and helps prevent it from entering the mouth, and therefore prevents it from being swallowed.

Before the specific endoscopic use of the embodiment is initiated, the tether is fed through the slit in the plate and the top of the elongated tether protector portion (as discussed—the post 408 preferably having ridge/rib 426) into a channel that is preferably has a ink-bottle-shaped cross section and positioned along the bottom of the incomplete cylinder like lower portion of the channel. an ink-bottle ending centrally within the cylindrical tether/cable protector. After the tether is fed into the bottom of the cross-sectionally ink-bottle shaped channel, the tether is free to move axially while, at the same time, being substantially secured against undesired transvers movements. The endoscopic capsule is next inserted into the open mouth of the patient, following which the bite guard is positioned so that the incisors or other teeth can bite and grip the post (the tether protector element of the embodiment) that at that moment substantially encases the tether.

Swallowing of the pill is next initiated and facilitated by drinking fluids such as water. Wide (preferably, bilateral) side-opening(s) of the front plate of the embodiment allow a straw or water-bottle with spout, or a glass or cup to be positioned so that the seated patient can drink water. This will help initiate swallowing and propel the capsule of the endoscopic tethered device distally through peristaltic movements of the esophageal musculature.

As the tether/cable can move freely in its semi-closed incomplete cylinder like bottom portion of the channel of the post of the mouth guard, peristalsis and/or gravity is used in a seated patient to advance the capsule distally; by applying gentle traction to the cable/tether, the position of the capsule can be directed for optimal imaging of a structure or lesion of interest. The incomplete cylinder like bottom portion of the channel allows the tether to be manually rotated about the axis of the channel to orient the capsule for viewing a target in different directions. Traction can also be optionally used to pull the capsule into the esophagus from the stomach and when the procedure is completed, the device can be removed from the mouth along with the bite-block, immediately after unhooking and releasing the elastic strap. Specifically, once the capsule is in the mouth, the patient or subject may be asked to open his or her mouth, and the bite-block, capsule, and elastic strap can be removed at the same time, after which the patient can close her/his mouth.

When the embodiment of the article of the invention is configured as a bite guard, it can be additionally or alternatively used with a tethered capsulated apparatus configured not for optical viewing but for manometry, pH studies, impedance studies, ultrasound, confocal microscopy, optical coherence tomography, or other processes during which a tethered device is required to be passed through a mouth of the subject.

While specific values chosen for this embodiment are recited, it is to be understood that, within the scope of the invention, the values of all of parameters may vary over wide ranges to suit different applications.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value.

The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes. Other specific examples of the meaning of the terms “substantially”, “about”, and/or “approximately” as applied to different practical situations may have been provided elsewhere in this disclosure.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

While the invention is described through the above-described exemplary embodiments, it will be understood by those of ordinary skill in the art that modifications to, and variations of, the illustrated embodiments may be made without departing from the inventive concepts disclosed herein. The disclosed aspects may be combined in ways not listed above. Accordingly, the invention should not be viewed as being limited to the disclosed embodiment(s).

What is claimed is:

1. An article of manufacture comprising:
a plate of a first material having a peripheral edge surface that defines a slit in the plate, the slit un-interruptingly extending from a peripheral portion of the plate to a central portion thereof, and
a post of a second material affixed, at a first end of the post, to a first side surface of said plate at the central portion thereof to protrude from said plate substantially transversely thereto, the post containing a channel formed therein throughout the post along an axis of the post and fluidly connecting the first end of the post and a second end of the post opposing the first end,
wherein the slit merges with and ends at a bottom of the channel such that an axis of the slit is substantially transverse to the axis of the post.

2. An article of manufacture according to claim 1, wherein said channel has a cross-section in a plane that is transverse to the axis, said cross-section including a first portion defined by two lines that are substantially parallel to one another and separated from one another by a first distance.

3. An article of manufacture according to claim 2, wherein said cross-section includes a second portion defined by an incomplete circle having a diameter that is larger than the first distance.

4. An article of manufacture according to claim 1, wherein a combination of the slit and the post is configured to fluidly connect the second end with a second surface of said plate, the second surface being opposite to the first surface.

5. An article of manufacture according to claim 1, wherein a plane containing the axis of the post and the axis of the slit is substantially normal to a perimeter of the plate.

6. An article of manufacture according to claim 5, wherein the article is substantially symmetric with respect to a plane in which both the axis of the post and the axis of the list lie.

7. An article of manufacture according to claim 1, wherein the post includes a ridge extending radially from the axis of the post at the second end.

8. An article of manufacture according to claim 1, containing at least one opening therethrough and/or containing first and second openings therethrough that are located substantially symmetrically with respect to the post.

9. An article of manufacture according to claim 1, wherein the plate is bent in a plane containing the axis of the post such that the post extends from the plate radially towards a center of curvature of a corresponding bend.

10. An article of manufacture according to claim 1, wherein the first material and the second material are the same.

11. An article of manufacture according to claim 1, wherein the plate and the post are co-molded.

12. An article of manufacture according to claim 1, configured as a mouth guard for use during an endoscopy procedure.

13. An article of manufacture according to claim 12, further comprising first and second means configured to assist in securing the article, inserted in a mouth of a user, with respect to a head of the user.

14. An article of manufacture according to claim 13, wherein the first means and the second means are configured as hooks for securing an elastic element thereto.

15. A method comprising:
passing the tether through a slit of an article of manufacture according to claim 1, and
positioning the tether along an axis of the post and in a channel of a post of the article of manufacture such that a first end of the tether is placed outside and next to a free end of the post while a second end of the tether is separated from the first end at least by a length of the post and a plate of the article of manufacture to which another end of the post is affixed.

16. A method according to claim 15, wherein said passing includes moving the tether along an axis of the slit towards the axis of the post.

17. A method according to claim 15, further comprising repositioning the tether within the channel along the axis of the post to vary an axial separation between the first end of the tether and the free end of the post while substantially not changing angular orientation of a portions of the tether contained within the channel.

18. A method according to claim 15, wherein said positioning includes positioning the tether along the axis of the post and in the channel of the post such that an optical camera at the first end of the tether is placed outside and net to the free end of the post while a control device, cooperated with the second end of the tether is separated from the optical camera at least by the length of the post and the plate.

19. A method according to claim 15, further comprising transmitting a fluid through at least one of the openings formed in the plate of the article of manufacture from a side of the plate corresponding to the second end of the tether to a side of the plate corresponding to the first end of the tether.
